(19) United States
(12) Patent Application Publication
(10) Pub. No.: US 2007/0160548 A1
(43) Pub. Date: Jul. 12, 2007

(54) Moisturizing sunless tanning composition

(75) Inventors: Grace Riccardi, Jersey City, NJ (US); Betty Pingol, Passaic, NJ (US); James P. Sanogueira, Suffern, NY (US); Olga V. Dueva-Koganov, White Plains, NY (US)

Correspondence Address: Charles N.J. Ruggiero;Ohlandt, Greeley, Ruggiero & Perle, L.L.P., 10th Floor, One Landmark Square, Stamford, CT, 06901-2682, US

(73) Assignee: Playtex Products, Inc.

(21) Appl. No.: 11/638,232

(22) Filed: Dec. 13, 2006

Related U.S. Application Data
(60) Provisional application No. 60/749,756, filed on Dec. 13, 2005.

(51) Int. Cl. A61K 8/60; A61K 8/49; A61K 8/35

(52) U.S. Cl. 424/59; 424/74

(57) ABSTRACT

There is provided a sunless tanning composition having a tanning active system and a moisturizing system. The moisturizing system is oil-free, which mitigates and/or eliminates skin irritation and/or tanning active occlusion.

### RELATED APPLICATION

This application claims the benefit of the earlier filed pending provisional patent application bearing Application Ser. No. 60/749,756 filed on Dec. 13, 2005. This application is incorporated in its entirety by reference herein.

### BACKGROUND OF THE DISCLOSURE

1. Field of the Disclosure

The present disclosure relates to a sunless tanning composition. More particularly, the present disclosure relates to a moisturizing sunless tanning composition.

2. Description of the Related Art

Products that are currently used for sunless tanning of the skin are based on the reaction of an active chemical present in the product with the skin's amino acids. Such chemicals are well known and include compounds having an aldehyde group, or compounds having a ketone group, such as, for example, dihydroxyacetone (DHA). As an example, DHA gives skin a brownish color by reacting with the amino acids present in the sebum and stratum corneum by a known mechanism, namely the Maillard reaction.

Unfortunately, the tanning active components can be very drying to the skin. To mitigate the drying effect, commercial sunless tanning products are formulated with added moisturizing components, such as mineral oil and petrolatum, to achieve the desired moisturizing properties.

Several drawbacks are associated with the use of mineral oil and/or petrolatum-based components in a sunless tanning product. Many consumers, especially those with sensitive skin, have adverse skin reactions to compositions with mineral oil and/or petrolatum-based components. Additionally, these oily components can impart a heavy, undesirable feel to the sunless tanning product.

Another potential drawback to compositions with mineral oil and/or petrolatum-based components is that a portion of the skin tanning active component(s) can be occluded in the composition when applied to the skin, thereby making it unavailable for immediate reaction with skin proteins or amino acids. This may result in an increase in time to achieve the desired artificial tan.

Thus, there is a need in the consumer products and cosmetic industry for a sunless tanning product that can effectively produce both a tanning and moisturizing effect on the skin, without skin irritation and or occlusion of tanning actives.

### SUMMARY OF THE DISCLOSURE

The present disclosure provides a sunless tanning composition having a tanning active system and a moisturizing system.

The present disclosure also provides such a sunless tanning composition where the moisturizing system is essentially oil-free.

The present disclosure further provides such a sunless tanning composition where the moisturizing system is oil-free.

The present disclosure still further provides a method of simultaneously sunless tanning and moisturizing having the step of topically applying to the skin an effective amount of a sunless tanning composition with a tanning active system and a moisturizing system.

The present disclosure yet further provides a sunless tanning product, such as, solution, emulsion, serum, lotion, skin cleanser, body wash, body scrub, bar soap, liquid soap, skin care preparation, foam, mousse, cream, pomade, balm, stick, gel, sprayable, non-sprayable, or any combinations thereof.

These and other advantages and benefits of the present disclosure are achieved by providing a sunless tanning composition having a tanning active system and a moisturizing system. The moisturizing system is preferably oil-free, which mitigates and/or eliminates skin irritation and/or tanning active occlusion.

### DESCRIPTION OF THE FIGURES

FIG. 1 is a graph depicting the mean conductance measurements versus time for two comparative formulations outside the scope of the present disclosure.

FIG. 2 is a graph depicting the net change in measured conductance over time from a baseline measurement for two comparative formulations outside the scope of the present disclosure.

FIG. 3 is a graph depicting the mean conductance measurements versus time of two compositions according to the present disclosure and one comparative composition.

FIG. 4 is a graph depicting the net change in measured conductance over time from a baseline measurement for two compositions according to the present disclosure and one comparative composition.

### DETAILED DESCRIPTION OF THE DISCLOSURE

The present disclosure provides a sunless tanning composition having a tanning active system in an amount effective for sunless tanning and a moisturizing system in an amount effective for moisturizing skin.

The sunless tanning composition according to the present disclosure is essentially oil-free. In one embodiment of the present disclosure, the sunless tanning composition is oil-free. The term “oil-free”, as used herein, is defined as free of mineral oil and/or petrolatum. By formulating the present compositions as oil-free, the compositions are non-irritating to sensitive skin, have an enhanced skin-feel when applied, and/or are less occlusive to the tanning actives.

The tanning active system of the present disclosure includes one or more sunless tanning actives for imparting a tan to the skin. Suitable tanning actives for use in the tanning active system of the present disclosure include, but are not limited to, DHA, erythrulose, melanin, mahakanni, or any combinations thereof. In one embodiment of the present disclosure, the tanning actives are DHA and erythrulose.

The tanning active system is present in the sunless tanning compositions of the present disclosure in an amount effective for imparting the desired tanning effect in the desired time. A suitable effective amount of the tanning active system is about 0.5 wt. % to about 10 wt. %, based on the total weight of the sunless tanning composition. In one embodiment of the present disclosure, the tanning active system is present in an amount about 1 wt. % to about 5 wt. %, based on the total weight of the composition. In yet another embodiment, the tanning active system is present in an amount about 1.5 wt. % to about 2.5 wt. %, based on the total weight of the composition.

The moisturizing system of the sunless tanning compositions is an important aspect of the present disclosure. The moisturizing system is formulated primarily to provide substantial moisturizing to the skin, including simultaneously with the tanning of the skin and even after the tanning has been completed. It has been found that substantial moisturizing to the skin can be achieved by the moisturizing system of the present disclosure without the inclusion of mineral oil and/or petrolatum. As a result, a composition that is less irritating to sensitive skin is formulated. Additionally, the absence of the mineral oil and petrolatum results in a less occlusive composition.

The moisturizing system of the present disclosure includes one or more moisturizing components suitable for providing the desired moisturizing effects in the absence of mineral oil and petrolatum. In one embodiment, the moisturizing system includes two or more moisturizing components. In yet another embodiment of the present disclosure, the moisturizing system includes at least three moisturizing components.

Suitable moisturizing components include, but are not limited to, one or more polyols, siloxanes, naturally occurring fats and oils, or any combinations thereof.

The one or more polyols that may be used as moisturizing components include, but are not limited to, glycerin, propylene glycol, butylene glycol, hexylene glycol, pentylene glycol, caprylyl glycol, sorbitol, or any combinations thereof.

The one or more siloxanes that may be used as moisturizing components in the present disclosure include, but are not limited to, dimethicone, cyclomethicone, phenyl trimethicone, phenyl dimethicone, cetyl dimethicone, stearyl dimethicone, amodimethicone, C₃₀₋₄₅ alkyl dimethicone, C₃₀₋₄₅ Alkyl Methicone, Cetearyl methicone, dimethicone copolyol, cyclopentasiloxane, or any combinations thereof.

The one or more naturally occurring fats and oils that may be used as moisturizing components in the present disclosure include, but are not limited to, shea butter, shea butter oil, cocoa butter, jojoba butter, aloe butter, olive butter, coconut oil, jojoba oil, olive oil, sunflower seed oil, meadowfoam seed oil, macadamia nut oil, sesame oil, borage seed oil, or any combinations thereof.

The moisturizing system of the present disclosure is present in the sunless tanning composition in an amount about 1 wt. % to about 15 wt. %, based on the total weight of the composition. In one embodiment, the moisturizing system is present in an amount about 2 wt. % to about 10 wt. %, based on the total weight of the composition. In yet another embodiment of the present disclosure, the moisturizing system is present in an amount about 4 wt. % to about 8 wt. %, based on the total weight of the composition.

In one embodiment of the present disclosure, it has been found that by including one or more polyols, one or more siloxanes, and one or more naturally occurring fats and oils in the moisturizing system, the resulting sunless tanning composition with this moisturizing system has the desired moisturizing properties, while being non-irritating and/or non-occlusive. Also, the composition may unexpectedly have an enhanced skin-feel.

In one embodiment of the present disclosure, the one or more polyols, one or more siloxanes, and one or more naturally occurring fats and oils should be present in a weight ratio of about 20:5:1 to about 7:2:1.

In yet another embodiment of the present disclosure the moisturizing system has dimethicone, glycerin and shea butter present in a ratio of about 14:4.4:1 to about 12:2.5:1. This particular embodiment, when used to formulate a sunless tanning composition according to the present disclosure, provides enhanced moisturizing properties to the composition.

The sunless tanning compositions according to the present disclosure may further include one or more amphoglycinates (amphoacetates). Suitable amphoglycinates include, but are not limited to sodium oliveamphoacetate, sodium sunflowerseed amphoacetate, sodium cocoabutter amphoacetate, sodium sesame amphoacetate, sodium sweetalmond amphoacetate, sodium caproamphoacetate, sodium capryloamphoacetate, sodium isostearoamphoacetate, sodium lauroamphoacetate, sodium myristoamphoacetate, sodium oleoamphoacetate, sodium palmamphoacetate, sodium peanutamphoacetate, sodium ricinoleoamphoacetate, sodium stearoamphoacetate, sodium tallowamphoacetate, sodium undecylenamphoacetate, sodium wheat germamphoacetate, sodium cocoyl glycinate (Amilite GCS-11, Ajinomoto), or any mixtures thereof. These compounds are generally available from a variety of sources or can be prepared by methods known in the art.

The amphoglycinate may be present in the sunless tanning composition in an amount about 1 wt. % to about 30 wt. %, based on the total weight of the composition. In one preferred embodiment, when amphoglycinate is present, a two-component system is used to keep the amphoglycinate separate from the sunless tanning active. Suitable two-component systems for use in the present disclosure are described in co-pending U.S. patent application Ser. No. 11/174,044 filed on Jul. 1, 2005, entitled “Sunless Tanning System That Optimizes Color Development,” the disclosure of which, as it relates to two-component systems, is incorporated by reference herein.

The sunless tanning composition of the present disclosure can further include a cosmetically acceptable vehicle, which can be present up to 98 wt. %, based on the total weight of the composition.

The sunless tanning composition may further include one or more additional components, including, but not limited to, sugars, keto-sugars, surface-active agents, polymers, softening agents, moisturizers, water-proofing agents, vitamins, sweet orange citroflavonoids, hesperitine, ozone stressed yeast lysate, Saccharomyces cerevisiae ferment filtrate lysate, methylsulfonyl methane (MSM), also known as dimethyl sulfone and methyl sulfone, UV filters, skin penetration agents, or any combinations thereof. These one or more additional components may be present in an amount up to about 30 wt. % of the total weight of the composition.

Suitable UV filters may include, but are not limited to, dibenzoylmethane, oxybenzone, sulisobenzone, dioxybenzone, menthyl anthranilate, para aminobenzoic acid (PABA) ester, benzophenone-3, butyldibenzoylmethane (Parsol 1789), dimethyl cinnamate, octyl methoxycinnamate, DEA methoxycinnamate, octocrylene, drometrizole trisiloxane, octyl salicylate, homomenthyl salicylate, octyl dimethyl PABA, TEA salicylate, 4-methyl benzilidene camphor, 3-benzylidene camphor, benzylidene camphor sulfonic acid ester, octyl triazone, phenyl benzimidazole sulfonic acid ester, terephthalydiene dicamphor sulfonic acid ester, di-t-butyl hydroxybenzylidene camphor, ethyl PABA, butylmethoxy dibenzoylmethane (avobenzone), terephthalydiene methylene bis-benzotriazoyltetramethylbutyl-phenol, diethylhexyl-2,6-naphthalate, bis-ethylhexyloxyphenol methoxyphenol triazine, hydroxy methylphenyl benzotriazole, methylene bis-benzotriazoyltetramethylbutylphenol, bis-ethylhexyloxyphenol methoxyphenol triazine, hydroxybenzophenone, a benzotriazole, a dibenzoyl methane, an oxanilide, a hydroxy cinnamate, oil dispersible titanium dioxide, oil dispersible zinc oxide, a silicone-anchored sunscreen, para aminobenzoic acid (PABA), salicylic acid, TEA salicylate, benzylidene camphor sulfonic acid, phenyl benzimidazole sulfonic acid, terephthalydiene dicamphor sulfonic acid, hydroxy cinnamic acid, any derivatives thereof, or any combinations thereof.

Other suitable additives may include, but are not limited to, antioxidants, such as, erythrobic acid, sodium metabisulfite, sodium sulfite, rosemary extract, tocopherol, a derivative of tocopherol including a tocotriene, carotene, a carotenoid, lutein or lutein ester, a carotenoid, a phenolic antioxidant, a bioflavonoid, a plant extract, or any combinations thereof;

keratolytic agents, such as, salicylic acid, resorcinol, peroxide of an organic acid, or any combinations thereof;

anti-inflammatory agents, such as, steroidal and non-steroidal anti-inflammatory agents, plant extracts that have demonstrated anti-inflammatory activity, or any combinations thereof;

vitamins, such as, Vitamin K, Vitamin C, retinol (vitamin A), tocopherol, or any combinations thereof;

emollients, such as, cetearyl octanoate, octyl palmitate, glyceryl monostearate, caprylic trigylceride, capric trigylceride, or any combinations thereof;

humectants, such as, hyaluronic acid, one or more derivatives of hyaluronic acid, or any combinations thereof;

skin penetration enhancers, such as, ozone, SEPA, butylene glycol, cis-isomer of an unsaturated fatty acid, or any combinations thereof;

emulsifiers, such as, glyceryl stearate, cetearyl alcohol, cetyl alcohol, PEG-40 stearate, or any combinations thereof;

thickening agents, such as, xanthan gum, carbomer, clay, hydroxyethyl cellulose, or any combinations thereof;

preservatives, such as, an alkyl paraben, an alcohol, salts of benzoic acid, salts of sorbic acid, or any combinations thereof;

colorants, such as, synthetic and natural colorants, henna, caramel, photo-chromic and thermo-chromic colorants and pigments, surface-treated or hydrophobically modified colorants, or any combinations thereof;

organic acids and their derivatives, such as citric acid, glycolic acid, glutamic acid, gluco Delta lactone, or any combinations thereof;

chelating agents, such as, disodium EDTA;

pH adjusters, such as, an acid, a base, a buffer, or any combinations thereof, to adjust and maintain the pH to about 3.0 to about 7.5;

fragrances;

proteins;

peptides; and

amino acids.

The sunless tanning compositions typically have a vehicle. The vehicle should be a cosmetically acceptable or suitable vehicle. In the context of the present disclosure, the term “cosmetically acceptable vehicle” or “suitable vehicle” refer to any vehicle for a drug, a cosmetic or a medicament that is suitable for use in direct, safe contact with human tissues.

The sunless tanning composition is applicable to a variety of personal care product forms including, but not limited to body wash, bar soap, liquid soap, skin care preparation, cream, foam, gel, lotion, solution, emulsion, pomade, mousse, balm, stick, pump spray, aerosol spray, or any combinations thereof.

The sunless tanning composition can be organic solvent based, water based or it can be an emulsion, including, but not limited to, an oil-in-water emulsion or a water-in-oil emulsion.

The present disclosure provides a method of simultaneously sunless tanning and moisturizing skin having the step of topically applying to the skin an effective amount of a sunless tanning composition having the tanning active system and moisturizing system according to the present disclosure.

The Examples that follow are illustrative of the present disclosure. The Examples should not be construed as being limiting in any manner whatsoever.

### EXAMPLES

Several sunless tanning compositions were formulated and/or commercially obtained for testing. Their components are detailed below in Table 1.

TABLE 1

| | Comparative | Comparative | | |
|---|---|---|---|---|
| INCI/CTFA NAME | Example 1 | Example 2 | Example 1 | Example 2 |
| Deionized Water | x | x | x | x |
| Mineral Oil | | x | | |
| Petrolatum | | x | | |
| Glycerin | | x | x | x |
| Caprylic/Capric Triglycerides | x | | x | x |
| Glyceryl Dilaurate | | x | | |
| Glyceryl Stearate/PEG-100 Stearate | x | | x | x |
| Cetyl Alcohol | x | | x | x |
| C12-15 Alkyl Benzoate | x | | x | x |
| Stearyl Alcohol | x | | x | x |
| Dihydroxyacetone | x | x | x | x |
| Dimethicone | | x | x | x |
| Cetearyl Alcohol | | x | | |
| Cetearyl Alcohol/Cocoglucoside | x | | x | x |
| Acrylates/C10-30 Alkyl Acrylate crosspolymer | | x | | |
| Trimethylpentanediol/Adipic Acid Copolymer | x | | x | x |
| Phenoxyethanol/Methylparaben/Butylparaben/ | x | x | x | x |
| Ethylparaben/Propylparaben/Isobutylparaben | | | | |
| Stearic Acid | x | x | x | x |
| Butyrospermum Parkii (Shea Butter) | | | x | x |
| Ceteareth-20 | | x | | |
| PEG-7 Glyceryl Cocoate | x | | x | x |
| Steareth-20 | x | | x | x |
| Steareth-2 | x | | x | x |
| Disodium EDTA | x | x | x | x |
| Erythrulose | x | x | x | x |
| Fragrance | x | x | x | x |
| Retinyl Palmitate/Tocopherol | x | x | x | x |
| Sodium Ascorbyl Phosphate | x | | x | x |
| Aloe Barbadensis Leaf Juice | x | | x | x |
| Caramel | x | x | x | x |
| Avocado Fruit Extract | | x | | |
| Oatmeal Extract | | x | | |
| Jojoba Seed Extract | | x | | |
| Calendula Officinalis Flower Extract | | x | | |
| Olive Fruit Oil | | x | | |
| Cyclopentasilozane | | x | | |
| Sodium Hydroxide | | x | | |
| DMDM Hydantoin | | x | | |
| BHT | | x | | |
| Titanium Dioxide | | x | | |
| Mica | | x | | |
| Citric Acid | x | x | x | x |

Comparative Example 1 is a daily sunless tanning formulation without added moisturizers. Comparative Example 2 is Jergens Natural Glow Daily Moisturizer for Medium Skin Tones, which is commercially available. The moisturizing components glycerin, shea butter, and dimethicone were added to Comparative Example 1 in two different ratios in order to formulate two compositions (Examples 1 and 2) according to the present disclosure. Example 1 includes about 6 wt. % glycerin, about 0.5 wt. % shea butter, and about 1.25 wt. % dimethicone. Example 2 includes about 3.5 wt. % glycerin, about 0.25 wt. % shea butter, and about 1.1 wt. % dimethicone.

An 8-hour arm hydration study was conducted with Comparative Examples 1 and 2. In conducting this study, Good Clinical Practices (GCP) and Good Laboratory Practices (GLP) guidelines as well as the COLIPA Efficacy Testing Guidelines were followed. Briefly, panelists reported to the test facility for Baseline measurements of each of the six volar forearm test sites with a Skicon-200 Conductance Meter. The test products were then applied to four of the six test sites (while the other two sites were left as non-treated controls). Approximately 2, 4 and 8 hours after application, conductance measurements were repeated. The panelists were sequestered at the facility for the duration of the study. Each candidate was instructed to stop the use of all moisturizing products on their arms and washed their arms with the provided Ivory bar soap during a 3 day pre-conditioning period prior to testing.

The selected panelists reported to the test facility and their arrivals were staggered. Assignment of panelist number was in order of their arrival. Each panelist reported to a PhD scientist who logged each panelist in and outlined six 5 cm by 5 cm test sites on the left and right volar forearms using a standard template (3 on each arm). The panelists were instructed to keep their arms air-exposed for the duration of this study and not to touch their arms during the study.

In this study, an IBS Skicon-200 Conductance Meter was employed equipped with a Measurement Technologies probe to further enhance its ability to measure changes in skin surface hydration. It was anticipated that moisturizers would lead to increased conductance values over time. As has been shown, most notably by Obata and Tagami [Obata, M. and Tagami, H. A rapid in vitro test to assess skin moisturizers. In: J. Soc. Cosmet. Chem., 41, 235-241 (July/August, 1990)], the ability of an alternating current to flow through the stratum corneum is an indirect measure of its water content. The value recorded which is expressed in units of millimho represents the AC conductance 5 seconds after placing the spring-loaded probe tip to the sample site. This timing interval is sufficiently long enough for the electronic circuits to stabilize in response to this change in conductance but short enough not to be influenced by an increased hydration at the probe tip due to its being occlusive and acting as a hindrance to the normal water loss at the test site.

Five conductance measurements from each site at Baseline (prior to treatment) were taken. After the Baseline measurements were completed, the panelists reported to the Treatment Technician, who treated four of the six sites, which were located on the volar forearms. Treatment of the appropriate test material to the designated site was done according to a randomization schedule. Two sites (1 central site on each arm) remained non-treated to serve as controls.

The Treatment Technician delivered 0.05 cc of the test material using a syringe to the appropriate mapped site and then used a finger cot to gently spread an even film of the product over the test area. The time required to complete the treatment on the sites was approximately 4 minutes. The instrument operator was not involved in the treatment aspects of this study so that his instrument readings were done in a blind fashion.

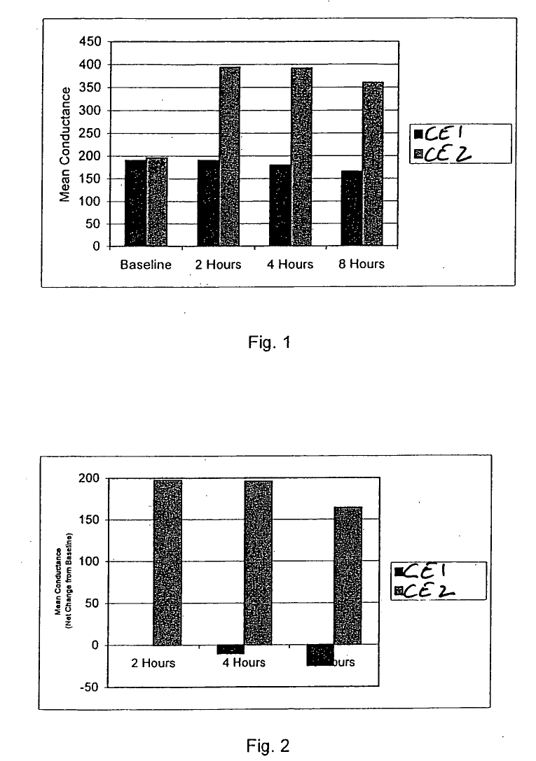

Conductance measurements were then taken at 2, 4 and 8 hours after the initial treatment. The average value was computed for each site after each measurement session. Results showed that Comparative Example 1 (CE1) provided no significant moisturizing effect on the skin and actually appeared to be drying after 4 hrs and significantly drying after 8 hrs (see FIGS. 1 and 2). This drying may be contributed to the sunless tanning actives in the formulation. Comparative Example 2 (CE2), with the added moisturizers glycerin, mineral oil, and petrolatum, showed significant moisturization levels after 2 hrs, 4 hrs, and 8 hrs, compared to Comparative Example 1.

A second moisturization test was conducted, which included Comparative Example 2 and Examples 1 and 2, according to the present disclosure. For this study, an 8-hour leg hydration protocol was used. This protocol was essentially identical to the above-described protocol, with the exception of the test site on the skin. Six test sites were outlined on the left and right outer calves, each measuring 5 cm by 5 cm using a standard template.

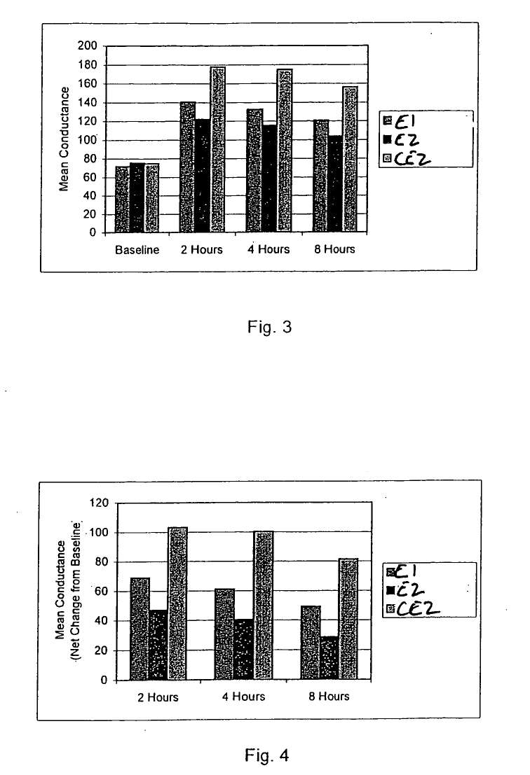

Results indicated that Example 1 (E1) resulted in a significant though moderate elevation in skin hydration after 8 hrs. Comparative Example 2 also resulted in a significant though moderate elevation in skin hydration after 8 hrs. Finally, Example 2 (E2) resulted in a significant though mild elevation in skin hydration after 8 hrs (see FIGS. 3 and 4). The test therefore concluded that the increases in skin hydration for all 3 formulations, whether they were mild or moderate, lasted longer than 8 hrs, which makes them long-lasting moisturizers. The moisturizing effect in Comparative Example 2 can be attributed to the occlusive ingredients—petrolatum and mineral oil in conjunction with glycerin. The moisturizing effects in Examples 1 and 2 according to the present disclosure, however, are generated without petrolatum and mineral oil.

The higher levels of moisturization for Example 1 compared to Example 2 were expected due to the increased amount of glycerin and shea butter. However, the higher levels of moisturizing agents in Example 1 actually unexpectedly produced a more pleasant skin feel. Example 1 was perceived to be less heavy and less greasy than Example 2. This was an unexpected result due to the fact that glycerin, shea butter, and dimethicone are substantive to the skin and can be perceived as having heavy or greasy feels.

It should be understood that the foregoing description is only illustrative of the present disclosure. Various alternatives and modifications can be devised by those skilled in the art without departing from the present disclosure. Accordingly, the present disclosure is intended to embrace all such alternatives, modifications and variations.

We claim:

1. A sunless tanning composition comprising:
a tanning active system; and
a moisturizing system,
wherein said composition is oil-free.

2. The sunless tanning composition of claim 1, wherein said tanning active system is present in the sunless tanning composition in an amount about 0.5 wt. % to about 10 wt. %, based on the total weight of the sunless tanning composition.

3. The sunless tanning composition of claim 1, wherein said tanning active system is present in the sunless tanning composition in an amount about 1 wt. % to about 5 wt. %, based on the total weight of the sunless tanning composition.

4. The sunless tanning composition of claim 1, wherein said tanning active system comprises one or more sunless tanning actives selected from the group consisting of DHA, erythrulose, melanin, mahakanni, and any combinations thereof.

5. The sunless tanning composition of claim 1, wherein said moisturizing system is present in the sunless tanning composition in an amount about 1 wt. % to about 15 wt. %, based on the total weight of the sunless tanning composition.

6. The sunless tanning composition of claim 1, wherein said moisturizing system is present in the sunless tanning composition in an amount about 2 wt. % to about 10 wt. %, based on the total weight of the sunless tanning composition.

7. The sunless tanning composition of claim 1, wherein said moisturizing system comprises two or more moisturizing components selected from the group consisting of polyols, siloxanes, naturally occurring fats and oils, and any combinations thereof.

8. The sunless tanning composition of claim 7, wherein said polyols are selected from the group consisting of glycerin, propylene glycol, butylene glycol, hexylene glycol, pentylene glycol, caprylyl glycol, sorbitol, and any combinations thereof.

9. The sunless tanning composition of claim 1, wherein said moisturizing system comprises at least three moisturizing components selected from the group consisting of polyols, siloxanes, naturally occurring fats and oils, and any combinations thereof.

10. The sunless tanning composition of claim 9, wherein said polyols are selected from the group consisting of glycerin, propylene glycol, butylene glycol, hexylene glycol, pentylene glycol, caprylyl glycol, sorbitol, and any combinations thereof.

11. The sunless tanning composition of claim 9, wherein said siloxanes are selected from the group consisting of dimethicone, cyclomethicone, phenyl trimethicone, phenyl dimethicone, cetyl dimethicone, stearyl dimethicone, amodimethicone, C30-45 alkyl dimethicone, C30-45 alkyl methicone, cetearyl methicone, dimethicone copolyol, cyclopentasiloxane, and any combinations thereof.

12. The sunless tanning composition of claim 9, wherein said naturally occurring fats and oils are selected from the group consisting of shea butter, shea butter oil, cocoa butter, jojoba butter, aloe butter, olive butter, coconut oil, jojoba oil, olive oil, sunflower seed oil, meadowfoam seed oil, macadamia nut oil, sesame oil, borage seed oil, and any combinations thereof.

13. The sunless tanning composition of claim 7, wherein said siloxanes are selected from the group consisting of dimethicone, cyclomethicone, phenyl trimethicone, phenyl dimethicone, cetyl dimethicone, stearyl dimethicone, amodimethicone, C30-45 alkyl dimethicone, C30-45 alkyl methicone, cetearyl methicone, dimethicone copolyol, cyclopentasiloxane, and any combinations thereof.

14. The sunless tanning composition of claim 7, wherein said naturally occurring fats and oils are selected from the group consisting of shea butter, shea butter oil, cocoa butter, jojoba butter, aloe butter, olive butter, coconut oil, jojoba oil, olive oil, sunflower seed oil, meadowfoam seed oil, macadamia nut oil, sesame oil, borage seed oil, and any combinations thereof.

15. A sunless tanning composition comprising:
a tanning active system; and
a moisturizing system comprising one or more polyols, one or more siloxanes, and one or more naturally occurring fats and oils,
wherein said composition is oil-free.

16. The sunless tanning composition of claim 15, wherein said one or more polyols, one or more siloxanes, and one or more naturally occurring fats and oils is present in the sunless tanning composition in a weight ratio of about 20:5:1 to about 7:2:1.

17. The sunless tanning composition of claim 15, wherein said one or more polyols are selected from the group consisting of glycerin, propylene glycol, butylene glycol, hexylene glycol, pentylene glycol, caprylyl glycol, sorbitol, and any combinations thereof.

18. The sunless tanning composition of claim 15, wherein said one or more siloxanes are selected from the group consisting of dimethicone, cyclomethicone, phenyl trimethicone, phenyl dimethicone, cetyl dimethicone, stearyl dimethicone, amodimethicone, C30-45 alkyl dimethicone, C30-45 alkyl methicone, cetearyl methicone, dimethicone copolyol, cyclopentasiloxane, and any combinations thereof.

19. The sunless tanning composition of claim 15, wherein said one or more naturally occurring fats and oils are selected from the group consisting of shea butter, shea butter oil, cocoa butter, jojoba butter, aloe butter, olive butter, coconut oil, jojoba oil, olive oil, sunflower seed oil, meadowfoam seed oil, macadamia nut oil, sesame oil, borage seed oil, and any combinations thereof.

20. A sunless tanning composition comprising:
a tanning active system; and
a moisturizing system comprising dimethicone, glycerin and shea butter present in a ratio of about 14:4.4:1 to about 12:2.5:1,
wherein said sunless tanning composition is oil-free.
